(19) United States
(12) Patent Application Publication
(10) Pub. No.: US 2014/0037774 A1
(43) Pub. Date: Feb. 6, 2014

(54) COMPOSITION COMPRISING THYLAKOIDS FOR DELAYING THE UPTAKE OF MOLECULES

(71) Applicants: Charlotte Erlanson Albertsson, Lund (SE); Per-Ake Albertsson, Lund (SE)

(72) Inventors: Charlotte Erlanson Albertsson, Lund (SE); Per-Ake Albertsson, Lund (SE)

(73) Assignee: THYLABISCO AB, Lund (SE)

(21) Appl. No.: 14/001,280

(22) PCT Filed: Feb. 24, 2012
(86) PCT No.: PCT/EP12/53181

§ 371 Date: Oct. 21, 2013

(30) Foreign Application Priority Data
Feb. 25, 2011 (SE) 1150176-4

(51) Int. Cl. A61K 36/21; A23L 1/30

(52) U.S. Cl. CPC A61K 36/21; A23L 1/3002
USPC 424/774

(57) ABSTRACT

Disclosed, is a composition comprising thylakoids, or parts thereof, for reducing the transport of molecules through the intestinal mucosa of the intestinal tract of a mammal.

### FIELD OF INVENTION

The present invention relates to use of composition for regulating the concentration in blood of various molecules by affecting their uptake in the gastro-intestinal system of a mammal. Further, the present invention relates to a method of reducing the transport of molecules through the intestinal mucosa of the intestinal tract of a mammal.

### BACKGROUND OF INVENTION

Hyperglycaemia is a major symptom in objects suffering from non-insulin dependent diabetes, the most prevalent disease affecting individuals with a Westernized life style (Yoon K H, Lee J H, Kim J W, Cho J H et al, Lancet 368: 1681-1688, 2006). Hyperglycaemia is further associated with coronary heart disease, obesity and chronic inflammation (Sinnaeve P R, Steg P G, Fox K A, et al. Archives Internal Medicine 169:402-409, 2009; Shoelson S E, Goldfine A B, Nature Medicine, 15: 373-374, 2009). In affected patients, elevated postprandial glucose is an early indicator of glucose intolerance or a pre-diabetes condition. The elevated postprandial glucose level is a more sensitive indicator of pre-diabetes than elevated fasting glucose levels. Treating patients who have early signs of hyperglycaemia, including elevated postprandial glucose level, with intensive glucose control is therefore fundamental to prevent or reduce later cardiovascular morbidity and mortality.

Other diseases affected by hyperglycaemia include genetic diseases such as schizophrenia and mental depression. These are triggered by production of advanced glycation end products following the reaction between elevated blood glucose levels and intrinsic proteins (Bengmark S, Journal of Parenteral and Enteral Nutrition, 31: 430-440, 2007).

A reduction of blood glucose levels is thus a desired aim. Management of hyperglycaemia disclosed in the art typically relies on:
reducing the dietary carbohydrate intake;
replacing the use of high-glycaemic carbohydrates by low-glycaemic carbohydrates; and/or
using hypoglycaemic agents that stimulate the uptake of glucose from the blood into peripheral cells.

Medical therapies to reduce hyperglycaemia in patient suffering from type 2 diabetes mellitus, also known as non-insulin dependent diabetes, typically involve insulin-dependent mechanisms and do thereby lose their effectiveness as pancreatic β-cell function declines (Smiley D, Umpierrez G E, Annals New York Academy. Science 1212: 1-11, 2010).

After digestion of dietary carbohydrates, glucose is taken up through the apical brush border membrane of the epithelial intestinal cells, mainly by the sodium-glucose linked transporter (SGLT1), to the enterocyte (Dyer J, Wood I S, Palejwala A et al Am J Physiol Gastrointest Liver Physiol 282: G241-248, 2002).

Control of the intestinal absorption of glucose would thus be of help to regulate levels of glucose in the blood. Accordingly, compositions affecting the intestinal absorption of glucose would be of interest.

It would further be of interest to be able to regulate the uptake of small as well as large molecules, such as saccharides, amino acids, peptides, and proteins, particularly in patients with disturbed intestinal barrier function.

### SUMMARY OF INVENTION

Consequently, the present invention seeks to mitigate, alleviate, eliminate or circumvent conditions associated with hyperglycaemia by providing a composition comprising thylakoids or parts thereof, for reducing the transport of molecules through the intestinal mucosa of the intestinal tract of a mammal and thereby delaying the uptake of molecules, such as saccharides, such as mono-, oligo- or polysaccaride, or proteins, or a mixture thereof, from the gastrointestinal tract, wherein said composition is to be administered to the intestinal tract of a mammal. Thylakoids are the photosynthetic membranes present in green leaves and green algae.

In an embodiment, the molecule is glucose.

Further, the present invention seeks to regulate the concentration of such molecules in blood by delaying their uptake, thereby preventing the development of their elevated postprandial concentration, for example reducing hyperglycemia.

Another aspect of the invention relates to the use of a composition comprising thylakoids or parts thereof, for delaying gastrointestinal uptake of molecules, such as glucose, and thereby reducing the peak of their concentration in blood. By reducing the intestinal uptake of glucose, development of diabetes and other diseases and disorders related to hyperglycaemia, such as such as diabetes mellitus, schizophrenia and mental depression may be prevented and/or treated.

In an embodiment, the thylakoids are enriched from green leaves or green algae.

The composition may be provided so that the dose of thylakoids is 2.4 mg to 24 mg thylakoids per kg body mass of the mammal.

The composition may be an oral pharmaceutical or nutraceutical composition comprising a physiologically tolerable oil-in-water emulsion comprising thylakoids.

In an embodiment, the pharmaceutical or nutraceutical composition comprises 25% (weight) thylakoids.

In some embodiments, the thylakoid composition utilized in the neutraceutical or pharmaceutical is a thylakoid concentrate. In preferred embodiments, the tylakoid concentrate contains concentrated thylakoid membranes. Suitable methods for preparing such concentrates are described in WO2010008333, which is incorporated by reference herein in its entirety. In some embodiments, the thylakoid composition, preferably a concentrate, utilized in the pharmaceutical or nutraceutical composition is characterized in having a chlorophyll a/b ratio of from about 2.0 to 4.0, preferably about 2.6 to 3.4, and most preferably about 2.9 to 3.4. In some embodiments, the thylakoid composition, preferably a concentrate, utilized in the pharmaceutical or nutraceutical composition is characterized in having a protein:chlorophyll ratio of from about 3.0 to about 10.0, preferably about 2.0 to 5.0, 3.0 to 5.0, or 6.0 to 8.0.

In some embodiments, the pharmaceutical or nutraceutical composition is characterized in having from about 8 to about 80 mg chlorophyll content per gram (dry weight, water content less than 6% (weight)), preferably from about 10 to about 50 mg, preferably about 10 mg to 30 mg, 20 mg to 40 mg, or 30 mg to 40 mg chlorophyll content per gram (dry weight).

In some embodiments, the pharmaceutical or nutraceutical composition is characterized in having from about 5 to about 50 g carbohydrate content per 100 g (dry weight, water content less than 6% (weight)), preferably about 10 g to 40 g per 100 g, preferably 15 g to 30 g, 20 g to 30 g or 30 g to 40 g per 100 g (dry weight),In some embodiments, the pharmaceutical or nutraceutical composition is characterized in having from about 10 to about 60 g protein content per 100 g (dry weight, water content less than 6% (weight)), preferably about 10 g to 40 g per 100 g, preferably 15 g to 30 g, 20 g to 30 g or 30 g to 40 g per 100 g (dry weight).

In an embodiment, the pharmaceutical or nutraceutical composition further comprises 25% (weight) oil.

The pharmaceutical or nutraceutical composition may be used in treatment by oral administration of a condition related to blood glucose levels.

The pharmaceutical or nutraceutical composition may also be used to enhance glucose metabolism, to improve blood glucose regulation, to improve pancreatic health, to lower appetite or to decrease body weight in a subject.

A further aspect of the invention relates to a composition comprising thylakoids, or parts thereof, for delaying the uptake of molecules, such as proteins, eg. ovalbumin, amino acids, peptides, oligosaccharide or polysaccharide, eg. dextran, in the intestinal tract of mammal.

In an embodiment, the thylakoids are enriched from green leaves, such as spinach, or green algae.

The composition may be provided so that the dose of thylakoids is 2.4 mg to 24 mg thylakoids per kg body mass of the mammal.

In an embodiment, the composition is administered orally.

The composition may preferably be a part of a foodstuff or a medicament.

The composition may be an oral pharmaceutical or nutraceutical composition comprising a physiologically tolerable oil-in-water emulsion comprising thylakoids.

In an embodiment, the pharmaceutical or nutraceutical composition comprises 25% (weight) thylakoids.

In some embodiments, the thylakoid composition utilized in the neutraceutical or pharmaceutical is a thylakoid concentrate. In preferred embodiments, the thylakoid concentrate contains concentrated thylakoid membranes. Suitable methods for preparing such concentrates are described in WO2010008333, which is incorporated by reference herein in its entirety. In some embodiments, the thylakoid composition, preferably a concentrate, utilized in the pharmaceutical or nutraceutical composition is characterized in having a chlorophyll a/b ratio of from about 2.0 to 4.0, preferably about 2.6 to 3.4, and most preferably about 2.9 to 3.4. In some embodiments, the thylakoid composition, preferably a concentrate, utilized in the pharmaceutical or nutraceutical composition is characterized in having a protein:chlorophyll ratio (w/w) of from about 3.0 to about 10.0, preferably about 2.0 to 5.0, 3.0 to 5.0, or 6.0 to 8.0.

In some embodiments, the pharmaceutical or nutraceutical composition is characterized in having from about 8 to about 80 mg chlorophyll content per gram (dry weight, water content less than 6% (weight)), preferably from about 10 to about 50 mg, preferably about 10 mg to 30 mg, 20 mg to 40 mg, or 30 mg to 40 mg chlorophyll content per gram (dry weight).

In some embodiments, the pharmaceutical or nutraceutical composition is characterized in having from about 5 to about 50 g carbohydrate content per 100 g (dry weight, water content less than 6% (weight)), preferably about 10 g to 40 g per 100 g, preferably 15 g to 30 g, 20 g to 30 g or 30 g to 40 g per 100 g (dry weight).

In some embodiments, the pharmaceutical or nutraceutical composition is characterized in having from about 10 to about 60 g protein content per 100 g (dry weight, water content less than 6% (weight)), preferably about 10 g to 40 g per 100 g, preferably 15 g to 30 g, 20 g to 30 g or 30 g to 40 g per 100 g (dry weight).

In an embodiment, the pharmaceutical or nutraceutical composition further comprises 25% (weight) oil.

The pharmaceutical or nutraceutical composition may be used in treatment by oral administration of a condition related to blood glucose levels.

The pharmaceutical or nutraceutical composition may also be used to enhance glucose metabolism, to improve blood glucose regulation, to improve pancreatic health, to lower appetite or to decrease body weight in a subject.

A further aspect of the invention relates to non-therapeutic use of a composition comprising thylakoids, or parts thereof, for reducing the transport of molecules through the intestinal mucosa of the intestinal tract of a mammal.

A further aspect of the invention relates to use of a composition comprising thylakoids, or parts thereof, for reducing glycemic index of foodstuff.

A further aspect of the invention relates to method of treatment of a condition in a mammalian subject by oral administration to said subject of an effective amount of a composition according embodiments of the invention.

The condition may be hyperglycemia, such as postprandial hyperglycemia, or a disease or a disorder associated with hyperglycemia, such as diabetes mellitus, schizophrenia and mental depression.

### BRIEF DESCRIPTION OF DRAWINGS

FIGS. 1 to 3 depict the effect of thylakoids on the time dependent passage of molecules through the rat (using the technique of the Ussing chamber (3, 4)). Especially,

FIG. 1 shows the time dependent passage of 3H-methyl-glucose through the rat mucosa in the absence of thylakoids (control) and in the absence of increasing concentration of thylakoids (1.2, 2.9, and 5.8 mg chlorophyll/ml),

FIG. 2 shows the time dependent passage of FITC-D4-dextran through the rat mucosa in the absence of thylakoids (control) and in the presence of increasing concentration of thylakoids (0.6, 1.2, 2.9, 5.8, and 8.7 mg chlorophyll/ml), and

FIG. 3 shows the time dependent passage of ovalbumin through the rat mucosa in the absence of thylakoids (control) and in the presence of increasing concentrations of thylakoids (0.6, 1.2, 2.9, 5.8, and 8.7 mg chlorophyll/ml).

FIG. 4 depicts an electron microscopy photography of thylakoids (arrows) attached to the mucosa layer of rat intestine.

FIG. 5 depicts the concentration of blood glucose in rats after oral intake of glucose alone (small tilted squares) and after oral intake of glucose together with thylakoids (large squares).

### DETAILED DESCRIPTION OF THE INVENTION

The present inventors have found that oral administration of a composition comprising thylakoids, or parts thereof, to a mammal reduces the glucose uptake in the intestine of the mammal.

Thylakoids are digested by the body, why they can be used without any toxic effects. However, thylakoids are digested more slowly than other cell compartments, such as mitochondria, which is beneficial.

Thus, such compositions may be used for the prevention and/or treatment of hyperglycemia, and especially postprandial hyperglycemia. Further, by reducing the glucose uptake in the intestine, the blood glucose levels may be lowered. By lowering the blood glucose levels insulin resistance, diabetes, cardiovascular disease, and inflammation may be prevented and/or treated. Further, conditions with a strong inflammatory component, such as mental depression and schizophrenia, may be greatly improved.

Thus, the present invention relates to the use of thylakoids, i.e. photosynthetic membranes from green leaves or green algae, or parts thereof, for the reduction of the transport of molecules, such as glucose, through the intestinal mucosa of the intestine. By reducing the transport of such molecules, the intestinal uptake of molecules is delayed. Thus, diseases caused by unbalanced concentration of the molecules, such as glucose, in the blood may be prevented and/or treated. The thylakoids, or parts thereof, used may be part of a pharmaceutical as well as a food composition.

An embodiment, relates to the use of a composition comprising thylakoids, or parts thereof, for preventing and/or treating hyperglycemia and especially postprandial hyperglycemia. In some embodiments, the compositions are used to promote healthy blood sugar (e.g., glucose) levels, to manage blood sugar levels, to support healthy blood sugar regulation and/or balance, to reduce tendency to insulin resistance, and combinations thereof.

An embodiment, relates to the use of a composition comprising thylakoids, or parts thereof, for preventing and/or treating a disease or a disorder associated with hyperglycemia, such as diabetes mellitus, schizophrenia and mental depression. Especially, said disease or a disorder associated with hyperglycemia may be diabetes mellitus.

Further, an embodiment relates to the use of a composition comprising thylakoids, or parts thereof, for delaying the glucose uptake in the intestine of a mammal.

Said thylakoids may origin from leaves of any photosynthesizing plants such as clover, rape, sugar beet, dandelion, Arabidopsis thaliana, maize, tobacco, sun flower, salad, Chenopodium, Atriplex, spinach, mangold, quinoa, kale and grasses or a mixture thereof. Preferably, the thylakoids originate from leaves of spinach.

The composition may be used for oral administration. Further, it may be part of a foodstuff or a medicament.

Thylakoids are the photosynthetic membranes of green leaves or green algae and they are responsible for the light reaction of photosynthesis. The preparation of a composition comprising thylakoids, or parts thereof, may be carried out in many different ways, as has been described in the literature (cf. Andreasson et al., Emek et al., and WO 2006/7132586 A1, incorporated herein by reference in its entirety). Preferably the composition comprises thylakoids, or parts thereof, isolated or at least enriched from green leaves or green algae.

Further, thylakoids, or parts thereof, may be included in foodstuff in many different ways since they are themselves food and they can also be included in different types of pharmaceutical formula as previously described in WO 2006/7132586 A1.

### EXAMPLE 1

### Measurement of the Transport Through Intestinal Mucosa Using the Method of Ussing (3)

Thylakoid membranes were prepared by isolation from spinach leaves according to Andreasson et al. (1).The concentration of thylakoids was expressed as mg chlorophyll/ml (Chl/ml) and determined by the method of Porra (cf. reference (2)) 1 mg chlorophyll corresponds to about 12 mg thylakoids (5).

### Measurement of Transport of Molecules Through Intestinal Mucosa Using the Ussing Method (3)

A total of eight female rats (220-280 g) were anaesthetized in the morning with isoflouran whereafter the proximal and distal parts of the small intestine were collected, rinsed and immediately immersed in room-tempered modified Krebs buffer (0×1 M NaCl/3 mM CaCl2/5×5 mM KC1/14 mM KH2PO4/29 mM NaHCO3/5×7 mM Na-pyruvate/7 mM Na-fumarate/5×7 mM Na-glutamate/13×4 mM glucose/pH 7.4) oxygenated with carbogen (95/5%, O2/CO2). The intestine was cut in 3 cm sections, opened along the mesentery and pinched to the nails of the pre-heated (37° C.) Ussing half-cell (Precision Instrument Design, Los Altos, Calif., USA), modified in accordance with Grass and Sweetana (4). The half-cell was connected to another half-cell, and thus the arrangement had two sides: one facing the mucosal side of the intestine and the other facing the serosal side, with an exposed intestinal area of 1×78 cm2. The Ussing chambers were filled with 5 ml Krebs buffer, kept at 37° C. and connected to a carbogen supply. All intestinal segments were mounted in the chambers within 30 minutes after the intestine was collected. The intestinal segments were considered viable for a minimum of two hours after mounted in the chambers (6).

At start of the experiment, the buffer was exchanged to fresh buffer in the serosal half-cell, and test solution in the mucosal half-cell. The test solution (5 ml) consisted of modified Krebs buffer supplemented with oleic acid (18:1n-9, 5.07 mM), NaTDC (5.2 g/l), and the marker molecules methyl-D glucose, 3-O-methyl 3H (190 Da, 1.85′10-2 GBq, 3.1 μl/ml,) or methyl-D glucose, 3-O-methyl 14C (190 Da, 1.85′10-3 GBq, 0.8 μl/ml), FITC-dextran (4 000 Da, 1 g/l) and ovalbumin (45 000 Da, 25 g/l). Thylakoid membranes—both non-treated and treated with trypsin—were also added to the test solution to get final concentrations of 1.2, 2.9 and 5.8 chl mg/ml. This concentration interval is obtainable by dosing from about 0.2 mg to about 2 mg chlorophyll per kg body mass of the subject to be treated, which corresponds to about 2.4 mg to about 24 mg isolated thylakoids per kg body mass of the subject to be treated. Serosal samples of 1 ml were collected every 20 minutes during 120 minutes, with replacement of the sampled volume by fresh buffer.

The abovementioned dosage is for a composition with a high purity of thylakoids, i.e. isolated thylakoids.

In some embodiments, the dosing of thylakoids is equivalent to from about 1.0 mg to about 2.0 mg chlorophyll per kg body mass of the subject to be treated, preferably from about 1.5 to about 2.0 mg chlorophyll per kg body mass of the subject to be treated. This corresponds to about 12 mg to about 24 mg isolated thylakoids per kg body mass of the subject to be treated, preferably from about 18 mg to about 24 mg isolated thylakoids per kg body mass of the subject to be treated.

In some embodiments, the thylakoids are not isolated, but only concentrated to a suitable level, such as characterized in having from about 8 to about 80 mg chlorophyll content per gram (dry weight, water content less than 6% (weight)) composition, preferably from about 10 to about 50 mg, preferably about 10 mg to 30 mg, 20 mg to 40 mg, or 30 mg to 40 mg chlorophyll content per gram (dry weight) composition.

In some embodiments, the pharmaceutical or nutraceutical composition is characterized in having from about 5 to about 50 g carbohydrate content per 100 g (dry weight, water content less than 6% (weight)), preferably about 10 g to 40 g per 100 g, preferably 15 g to 30 g, 20 g to 30 g or 30 g to 40 g per 100 g (dry weight),In some embodiments, the pharmaceutical or nutraceutical composition is characterized in having from about 10 to about 60 g protein content per 100 g (dry weight, water content less than 6% (weight)), preferably about 10 g to 40 g per 100 g, preferably 15 g to 30 g, 20 g to 30 g or 30 g to 40 g per 100 g (dry weight).

In some embodiments, the thylakoids are concentrated so that the chlorophyll content is from about 8 mg to 80 mg chlorophyll per g composition (dry weight).

In some embodiments, the total daily does of the pharmaceutical or nutraceutical composition is from about 2 g to about 10 g.

The amount of radio labelled methyl-glucose in 0.5 -ml serosal samples was measured in a beta counter (Scint TriCarb Liquid Scintillation Analyzer 2100TR, Perkin Elmer, Boston, Mass., USA), after mixing the serosal sample with 5 ml liquid scintillation cocktail. FITC-dextran was quantified by fluorescence spectrophotometry (CytoFlour™ 2300, Millipore Corporation, Bedford, Mass., USA) at an excitation wavelength of 480 nm and an emission wavelength of 520 nm, where FITC-dextran dissolved in modified Krebs buffer was used as standard. Quantification of ovalbumin was performed by electro immunoassay (20) using a specific antiserum to ovalbumin (A6075) with purified ovalbumin (A-5503) as standard.

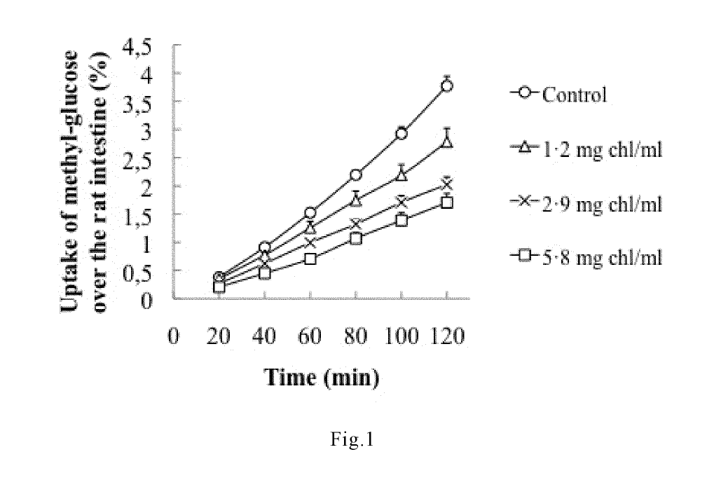

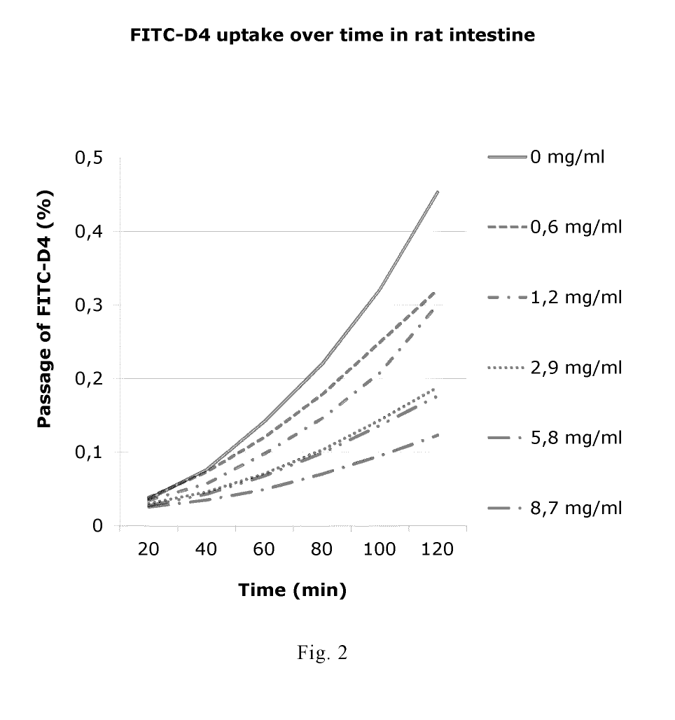

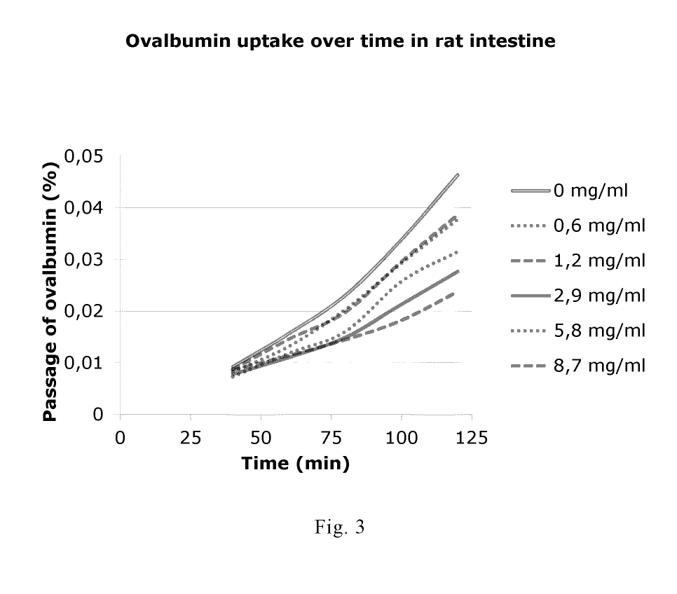

The results of the using experiments are shown in FIGS. 1 to 3. They show that thylakoids reduce the passage of all three molecules, representing different types of molecules, monosaccharides, polysaccharide and proteins, through the mucosa in a dose dependent way.

Following every experiment, a sample from the serosal half-cell was collected and analysed for chlorophyll content to investigate a possible thylakoid migration through the intestinal wall. No such migration was found.

### Electron Microscopy

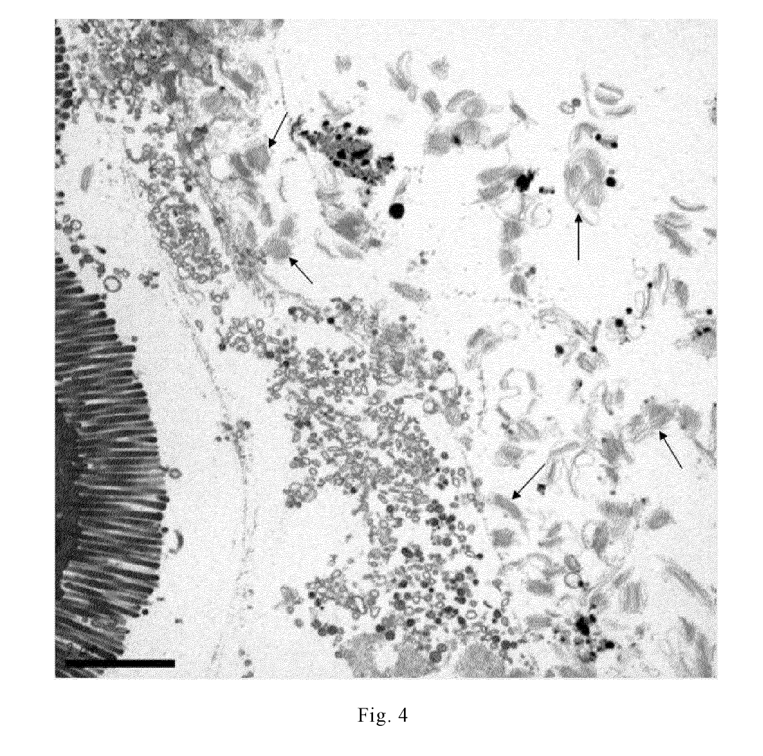

Intestinal segments incubated for 120 minutes in Ussing chambers in the absence of oleic acid and NaTDC but with thylakoid membranes (2.9 mg chl/ml) were fixed with 2.5% glutaraldehyde in 0.15M cacodylate buffer and embedded in Epon, and finally stained in 3% (v/v) uranyl acetate and lead citrate. FIG. 4 shows an electron microscope picture of thylakoids attached to the mucosa layer after an Ussing experiment.

Without being bound to any theory, it is believed that the retardation of the passage of molecules through the mucosa layer may be explained by the fact that the thylakoids seems to bind to the mucosa layer as shown by FIG. 4. A visual inspection of the mucosa layer seems to confirm this. It is hypothesized that this binding may form a thylakoid layer acting as a barrier on the surface of the mucosa. This barrier may constitute a sterical hindrance bale to retard the passage of molecules, both small and large, through the mucosa as shown by FIGS. 1 to 3.

### EXAMPLE 2

### Glucose Uptake by Rats In Vivo

Thylakoid membranes were prepared as described in EXAMPLE 1. Rats were of the Sprague-Dawley stock (Mole: SPRD Han).

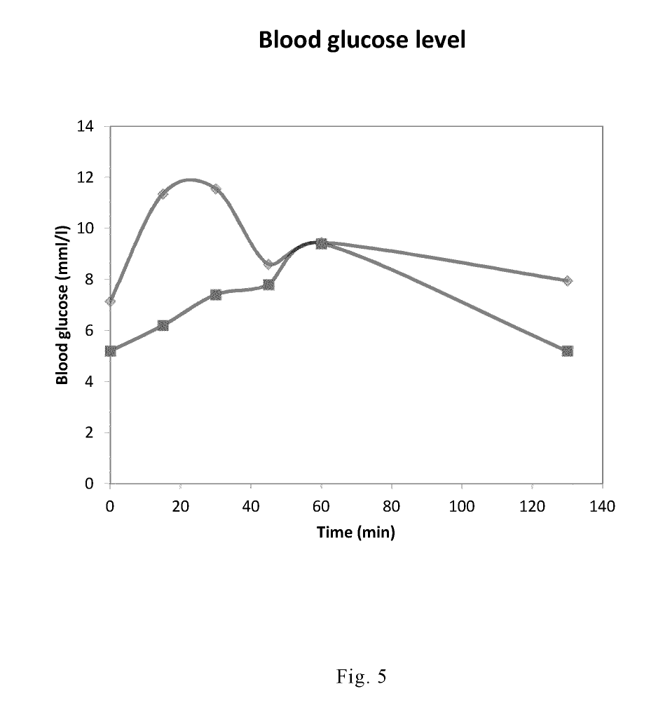

A rat was fed an emulsion of 2.5 g rapeseed oil in 5 g water (control) and a second rat was fed an emulsion of 2.5 g rapeseed oil in 5 g water plus 2.5 g thylakoids via bolustube. After 2.5 hours both rats were fed each 3 mL 15% glucose solution. Blood glucose was then measured at different times during 2 hours. The results are shown in Table 1 and FIG. 5. As can be seen there is a significant reduction of the blood glucose over time when thylakoids are co-administered.

An oil-in-water emulsion is advantageous, since it reduces the rate, with which thylakoids are digested by the body.

TABLE 1
Blood glucose concentration (mmol/L) at different times (min)
after consumption of glucose with (Thylakoids) or without
(Control) thykalkoids in the food.

| Time | Control | Thylakoids |
|---|---|---|
| 0 | 7.15 | 5.2 |
| 15 | 11.35 | 6.2 |
| 30 | 11.55 | 7.4 |
| 45 | 8.6 | 7.8 |
| 60 | 9.49 | 9.4 |

### REFERENCES

1. Andreasson, E., Svensson, P. & Weibull, C. & Albertsson, P. A. (1988) Separation and characterization of stroma and grana membranes—evidence for heterogeneity in antenna size of both Photosystem I and Photosystem II. Biochimica et Biophysica Acta. 936, 339-350.
2. Emek, S. C., Szilagyi, A., Akerlund, H. E., Albertsson, P. A., Köhnke, R., Holm, A. & Erlanson-Albertsson, C. (2010) A large scale method for preparation of plant thylakoids for use in body weight regulation. Prep. Biochem. Biotechnol. 40, 13-27.
3. Ussing H H, Zerahn K (1951) Active transport of sodium as the source of electric current in the short-circuited isolated frog skin. Acta Physiol. Scand. 23, 110-127.
4. Grass G, Sweetana S (1988) In vitro measurement of gastrointestinal tissue permeability using a new diffusion cell. Pharm. Res. 5, 372-376.
5. Flores S, Graan T, Ort D R (1983) Measurement of the permeability of the chloroplast thylakoid membrane to amine buffers. Photobiochem. Photobiophys. 6, 293-304
6. Pantzar N, Westrom B R, Luts A et al. (1993) Regional Small-Intestinal Permeability in Vitro to Different-Sized Dextrans and Proteins in the Rat. Scand. J. Gastroenterol. 28, 205-211.

1. A method of reducing and/or delaying the transport of glucose molecules through the intestinal tract of a mammal comprising administering to said mammal an effective amount of a composition comprising thylakoids, or parts thereof, enriched from green leaves or green algae, wherein said administration is to the intestinal tract of a mammal.

2. (canceled)

3. (canceled)

4. The method according to claim 1, wherein hyperglycemia is prevented by said reducing and/or delaying the transport of glucose through the intestinal mucosa of the intestinal tract of a mammal.

5-7. (canceled)

8. The method according to claim 1, wherein the thylakoids are concentrated so that the chlorophyll content is from about 8 mg to 80 mg chlorophyll per g composition (dry weight).

9. The method according to claim 1, wherein the composition is provided so that the dose of thylakoids is 2.4 mg to 24 mg thylakoids per kg body mass of the mammal

10. (canceled)

11. (canceled)

12. (canceled)

13. (canceled)

14. The method claim 1, wherein said composition is administered orally.

15. The method claim 1, wherein said composition is part of a foodstuff or a medicament.

16. The method claim 1, wherein said thylakoids, or parts thereof, are enriched from spinach.

17. The method according to claim 1, wherein the composition is an oral pharmaceutical or nutraceutical composition comprising a physiologically tolerable oil-in-water emulsion comprising thylakoids.

18. The method according to claim 17, wherein said composition comprises 25% (weight) thylakoids and about 25% (weight) oil.

19. (canceled)

20. The method claim 17, wherein the transport of glucose through the intestinal mucosa of the intestinal tract of a mammal is reduced and/or delayed to treat a condition related to blood glucose levels by oral administration of the composition.

21. The method claim 17, wherein the transport of glucose through the intestinal mucosa of the intestinal tract of a mammal is reduced and/or delayed to promote healthy blood sugar, to manage blood sugar levels, to support healthy blood sugar regulation, to enhance glucose metabolism, to improve blood glucose regulation, to improve pancreatic health, and combinations thereof.

22. (canceled)

23. A method of reducing the glycemic index of an initial foodstuff, comprising the step of adding a composition comprising thylakoids, or parts thereof, enriched from green leaves or green algae to the initial foodstuff to provide a modified foodstuff so that the glycemic index of the modified foodstuff is reduced as compared to the initial foodstuff when ingested by a mammal.

24. (canceled)

25. (canceled)

26. The method according to claim 23, wherein in the composition comprising the thylakoids are concentrated so that the chlorophyll content is from about 8 mg to 80 mg chlorophyll per g composition (dry weight).

27. The method according to claim 23, wherein said thylakoids, or parts thereof, are enriched from spinach.

28. The method according to claim 23, wherein the composition is an oral nutraceutical composition comprising a physiologically tolerable oil-in-water emulsion comprising thylakoids.

29. The method according to claim 28, wherein said composition further comprises 25% (weight) thylakoids and 25% (weight) oil.

30. A method of promoting healthy blood sugar, managing blood sugar levels, supporting healthy blood sugar regulation, enhancing glucose metabolism, improving blood glucose regulation, and/or improving pancreatic health in a subject comprising administering to said subject an effective amount of a concentrated thylakoid composition comprising from about 8 mg to 80 mg chlorophyll per g composition (dry weight).

31. The method of claim 30, wherein said effective amount of said concentrated thylakoid composition reduces and/or delays the transport of glucose molecules through the intestinal tract of a mammal.

32. The method according to claim 30, wherein the composition is provided so that the dose of thylakoids is 2.4 mg to 24 mg thylakoids per kg body mass of the mammal.

33. The method according to 30, wherein said composition is administered orally.

34. The method according to claim 30, wherein said composition is part of a foodstuff or a medicament.

35. The method according to claim 30, wherein said thylakoids, or parts thereof, are enriched from spinach.

36. The method according to claim 30, wherein the composition is an oral pharmaceutical or nutraceutical composition comprising a physiologically tolerable oil-in-water emulsion comprising thylakoids.

37. The method according to claim 36, wherein said composition further comprises 25% (weight) thylakoids and about 25% (weight) oil.
